(19) United States
(12) Patent Application Publication
(10) Pub. No.: US 2013/0172430 A1
(43) Pub. Date: Jul. 4, 2013

(54) PROGNOSIS AND TREATMENT OF BREAST CANCER

(71) Applicants: Michael P. Lisanti, Philadelphia, PA (US); Federica Sotgia, Philadelphia, PA (US)

(72) Inventors: Michael P. Lisanti, Philadelphia, PA (US); Federica Sotgia, Philadelphia, PA (US)

(21) Appl. No.: 13/314,732

(22) Filed: Dec. 8, 2011

Related U.S. Application Data
(60) Provisional application No. 61/421,807, filed on Dec. 10, 2010.

(51) Int. Cl. C12Q 1/68; A61K 45/00; G01N 30/00; C12Q 1/04

(52) U.S. Cl. CPC C12Q 1/6886; C12Q 1/04; A61K 45/00; G01N 30/00
USPC 514/789; 506/9; 435/34; 73/23.35; 73/61.52

(57) ABSTRACT

Methods of prognosis and monitoring of breast cancer include determining the level of one or more of the markers comprising asymmetric dimethyl arginine (ADMA), beta-hydroxybutyrate (BHB) and microRNA-31 (miR-31) in patient samples. An increased level is correlated with poor prognosis. Breast cancer is treated by administration of one or more inhibitors of ADMA and/or BHB.

### CROSS-REFERENCE TO RELATED APPLICATION

This application claims the filing date benefit of U.S. Provisional Application No. 61/421,807, filed Dec. 10, 2010 the entire disclosure of which is incorporated herein by reference.

### FIELD OF THE INVENTION

The present invention relates to the prognosis and treatment of breast cancer.

### BACKGROUND OF THE INVENTION

The loss of stromal Cav-1 in the tumor associated fibroblast compartment was previously identified as a critical event in the progression of human breast cancers (Mercier et al., Cancer Biol Ther 2008; 7:1212-25; Sotgia et al., Am J Pathol 2009; 174:746-61; Witkiewicz et al., Am J Pathol 2009; 174:2023-34). More specifically, a loss of stromal Cav-1 is associated with early tumor recurrence, lymph node metastasis and tamoxifen-resistance, resulting in poor clinical outcome (Mercier et al. supra) Similar results were obtained with DCIS and prostate cancer patients (Di Vizio et al., Cell Cycle 2009; 8:2420-4) indicating that a loss of stromal Cav-1 in cancer-associated fibroblasts is tightly associated with tumor progression and metastasis.

These findings have now been replicated in several independent patient cohorts (Witkiewicz et al., Cell Cycle 2009; 8:1654-8; Sloan et al., Am J Pathol 2009; 174:2035-43), and also extended to other more lethal forms of breast cancer, such as the triple-negative and basal-like breast cancer sub-types (Witkiewicz et al., Cancer Biol Ther 2010; 10:133-143). For example, in triple-negative breast cancers, patients with high stromal Cav-1 have a 75.5% survival rate at 12 years, while patients with an absence of stromal Cav-1 have a survival rate of less than 10% at 5 years post-diagnosis (Witkiewicz et al., supra).

Genetic ablation of Cav-1 has been shown to be sufficient to confer the cancer-associated fibroblast phenotype, and Cav-1 (−/−) mammary fibroblasts provide a cell culture model for breast cancer-associated fibroblasts (Pavlides et al., Cell Cycle 2009; 8:3984-4001). Accordingly, the mammary fat pad of Cav-1 (−/−) null mice serves as a pre-clinical model for a tumor-microenvironment.

What is needed are informative biomarkers for prognosis of breast cancer, which may be used to identify high-risk breast cancer patients at time of initial diagnosis, or for treatment stratification and/or for evaluating therapeutic efficacy during anti-cancer therapy.

What is further needed is new treatment modalities for breast cancer.

### SUMMARY OF THE INVENTION

A prognostic method for breast cancer in a subject is provided. The method comprises (a) determining the level of at least one of the following in a test sample from a subject: (i) asymmetric dimethyl arginine (ADMA); (ii) beta-hydroxybutyrate (BHB); and (iii) miR-31; and (b) comparing the level of at least one of (i) asymmetric dimethyl arginine (ADMA), (ii) beta-hydroxybutyrate (BHB), and (iii) miR-31 in the test sample to the level of in a control sample, wherein an elevated level of at least one of (i) asymmetric dimethyl arginine (ADMA), (ii) beta-hydroxybutyrate (BHB), and (iii) miR-31 in the test sample relative to the level in the control sample is a prognostic indicator of the course of breast cancer disease in said subject. An increased level of (i), (ii) or (iii) is correlated with poor prognosis.

In another embodiment, a method of monitoring the progression of breast cancer in a subject is provided. The method comprises (a) obtaining a first sample from a subject at a first time point and a second sample from said subject at a second time point; (b) determining the level of at least one of the following in the first and second samples: (i) asymmetric dimethyl arginine (ADMA); (ii) beta-hydroxybutyrate (BHB); and (iii) miR-31; and (c) comparing the level of said at least one of (i) asymmetric dimethyl arginine, (ii) beta-hydroxybutyrate (BHB), and (iii) miR-31 in the first sample to the level in said second sample, wherein an elevated level of at least one of (i) asymmetric dimethyl arginine, (ii) beta-hydroxybutyrate (BHB), and (iii) miR-31 in the second sample relative to the level in the first sample is an indication that the cancer has progressed in the subject.

In one embodiment, the level of ADMA is determined. In another embodiment, the level of BHB is determined. In yet another embodiment, the level of miR-31 is determined.

In certain embodiments, the sample comprises serum or plasma. In other embodiments, the sample comprises excised tumor tissue.

A method for treating breast cancer in a breast cancer patient in need of such treatment is provided. The method comprises administering to an effective amount of an inhibitor of ADMA or BHB an inhibitor of BHB to the patient. In another embodiment, an effective amount of an inhibitor of an enzyme associated with (1) ADMA production, (2) ketone production or (3) ketone re-utilization is administered.

In one embodiment, an effective amount of an inhibitor of ADMA is administered to the patient. In another embodiment, an effective amount of an inhibitor of BHB is administered to the patient.

### BRIEF DESCRIPTION OF THE DRAWINGS

FIG. 1 is a plot of the metabolic analysis of ADMA and BHB in the mammary fat pad and lung tissue of Cav-1 (−/−) null mouse

### DEFINITIONS AND ABBREVIATIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one elements.

The term “about” will be understood by persons of ordinary skill in the art and will vary to some extent depending on the context in which it is used. As used herein, “about” is meant to encompass variations of ±20% or ±10%, more preferably ±5%, even more preferably ±1%, and still more preferably ±0.1%.

“ADMA” means asymmetric dimethyl arginine.

“BHB” means beta-hydroxybutyrate, also known as 3-hydroxybutrate, beta-hydroxybutyric acid and 3-hydroxybutyric acid.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies that may be used in the practice of the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, New York; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “breast cancer” as used herein is defined as cancer which originates in the breast.

The term “control” or “reference standard” describes a material comprising a level of a target molecule, such that the control or reference standard may serve as a comparator against which the level of the target molecule in a patient sample can be compared.

The term “inhibitor” as used herein with respect to a target molecule refers to an agent that inhibits the biological effect of the target molecule.

As used herein, an “instructional material” includes a publication, a recording, a diagram, or any other medium of expression, which can be used to communicate the usefulness of the invention in the kit for determining the progression of a disease. The instructional material of the kit of the invention may, for example, be affixed to a container, which contains a reagent of the invention or be shipped together with a container, which contains a reagent. Alternatively, the instructional material may be shipped separately from the container with the intention that the instructional material and the reagent be used cooperatively by the recipient.

“Measuring” or “measurement,” or alternatively “detecting” or “detection,” or alternatively “determining” or “determine” means assessing the presence, absence, quantity or amount of either a given substance within a sample, including the derivation of qualitative or quantitative concentration levels of such substances.

The term “prognosis,” as used herein, refers to a prediction of the probable outcome and course of a disease or condition.

“Sample” or “biological sample” as used herein means a biological material that contains a target molecule under assay for determination of its level. The sample may contain any biological material. It may comprise cellular and/or non-cellular material.

The term “solid support,” “support,” and “substrate” as used herein are used interchangeably and refer to a material or group of materials having a rigid or semi-rigid surface or surfaces. In one embodiment, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

“Specifically binds” as used herein in the context of an antibody or an aptamer refers to antibody or aptamer binding to a predetermined antigen with a preference that enables the antibody to be used to distinguish the antigen from others to an extent that permits the detection of the target antigens described herein.

The term “therapeutically effective amount” or “effective amount” means the amount of a subject compound or combination that will elicit the biological or medical response of a tissue, system, animal or human that is being sought.

As envisioned in the present invention with respect to the disclosed compositions of matter and methods, in one aspect the embodiments of the invention comprise the components and/or steps disclosed therein. In another aspect, the embodiments of the invention consist essentially of the components and/or steps disclosed therein. In yet another aspect, the embodiments of the invention consist of the components and/or steps disclosed therein.

### DETAILED DESCRIPTION OF THE INVENTION

The methods described herein rely on assessing the level of ADMA, BHB or miR-31 as a biomarker for determining a breast cancer prognosis, or for monitoring the progression of breast cancer, in an individual afflicted with breast cancer.

Using metabolomic analysis, we have identified ADMA and BHB as the two major metabolites which increased in Cav-1 (−/−) null mammary fat pads and lung tissue. The mammary fat pad of Cav-1 (−/−) null mice serves as a pre-clinical model for a tumor-microenvironment, as a loss of stromal Cav-1 is associated with early tumor recurrence, lymph node metastasis and tamoxifen-resistance, resulting in poor clinical outcome (Mercier et al. supra); genetic ablation of Cav-1 has been shown to be sufficient to confer the cancer-associated fibroblast phenotype (Pavlides et al., supra).

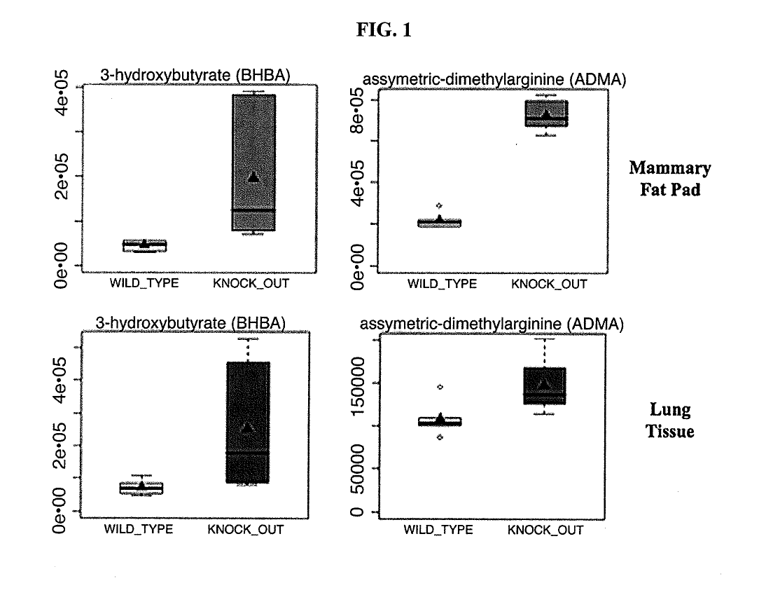

As described herein, metabolomic analysis of Cav-1 (−/−) null tissues from mammary fat pads has now evidenced an increase in the level of ADMA of 3.3.fold, and an increase in the level of BHB of 4.3 fold in Cav-1 (−/−) animals. These results from the mammary fat pad were confirmed with lung tissue from the Cav-1 (−/−) animals. ADMA and BHB were significantly elevated in lung tissue, consistent with the idea that Cav-1 (−/−) null tissues are undergoing (1) oxidative stress and (2) mitochondrial dysfunction. Box plots for ADMA and BHB are shown in FIG. 1.

ADMA is a marker of endothelial dysfunction and oxidative stress; it can also drive oxidative stress, as it functions as an uncoupler of NOS family member, inhibiting the production NO and producing superoxide instead. BHBA is a ketone body known to be a marker of mitochondrial dysfunction. Oxidative stress induces mitochondrial dysfunction and visa versa, driving autophagy and mitophagy. Without wishing to be bound by any theory, it is believed that these two metabolites (ADMA and BHB) are reflective of oxidative stress and mitochondrial dysfunction in Cav-1 (−/−) stromal cells.

Since ADMA and BHB emerged as the two most important metabolites that were increased in our metabolomic analysis, we validated that the enzymes responsible for their production were transcriptionally increased both in Cav-1 (−/−) stromal cells and the tumor stroma isolated from human breast cancers. Both transcriptional profiles from Cav-1 (−/−) null stromal cells and human breast cancer tumor stroma were analyzed in parallel and are presented in Example 4, Table 4. For this purpose, we analyzed the mRNA expression of the genes involved in ADMA production (PRMT gene family members) and degradation (DDAH1/2), as well as nitric oxide (NO) related genes, as ADMA drives NOS uncoupling and the production of superoxide, instead of NO. Using this approach, we observed that the genes involved in both ADMA production (PRMT2/7/8) and degradation (DDAH1/2), as well as nitric oxide production (NOS1/2/3 or NOS trafficking), are all transcriptionally overexpressed, both in human tumor stroma and in Cav-1 (−/−) stromal cells.

As BHB is a ketone body, we analyzed ketone metabolism in Cav-1 (−/−)cells. For this purpose, we analyzed the transcriptional profiles of the genes associated with both ketone production (ACYL, HMGCS1/2, HMGCL, HMGCLL1 and BDH1/2) and ketone re-utilization (ACAT1/2 and OXCT1/2). Only the genes associated with ketone production, but not ketone re-utilization were associated with human tumor stroma (Example 4, Table 4). This is consistent with the prediction that, as the epithelial cancer cells should express the genes associated with ketone re-utilization, so that they can re-use BHB as an energy source for mitochondrial oxidative metabolism. Also, many of the stromal genes involved in ketone production are specifically associated with tumor recurrence (ACLY, HMGCS2, HMGCLL1 and BDH1) and/or metastasis (BDH2). Many of these ketone production genes are also transcriptionally overexpressed in Cav-1 (−/−) stromal cells, consistent with our current metabolic analysis.

ADMA is a catabolic breakdown product released from methylated proteins after their proteolytic degradation. It is known to be strongly associated with endothelial cell dysfunction and oxidative stress. In addition, it also has biological activity and can enhance and propagate the effects of oxidative stress. ADMA is both a marker of oxidative stress and actively generates more oxidative stress. Furthermore, ADMA changes the location of eNOS and directly targets the enzyme to mitochondria, where it produces superoxide. ADMA leads to irreversible oxidative damage within mitochondria, necessitating their destruction by mitophagy. This, in turn, provides a mechanism for turning on aerobic glycolysis, so that the stromal cells will produce energy to ensure their own survival. However, aerobic glycolysis in the stroma releases both lactate and pyruvate, which can be used by epithelial cancer cells undergoing TCA-based oxidative metabolism, thereby providing paracrine energy for tumor growth.

Stromal ketone production, and BHB in particular, also is believed to play a strong pathogenic role. Ketone production is a well-established marker of mitochondrial dysfunction. BHB can be transferred directly from stromal cancer-associated fibroblasts to epithelial cancer cells via moncarboxylic acid transporters (MCTs), without any energy expenditure. Ketones are a “super-fuel” for mitochondria, producing more energy than lactate/pyruvate, and simultaneously decreasing oxygen consumption. Without wishing to be bound by any theory, stromal ketone production could obviate the need for tumor angiogenesis. Once ketones are produced and released from stromal cells, they could then be re-utilized by epithelial cancer cells, where they could directly enter the TCA cycle.

Without wishing to be bound by any theory, it is believed that the production of BBH results from acetyl-CoA derived from pyruvate, via pyruvate dehydrogenase (PDH) and not from the beta-oxidation of fatty acids, because Cav-1 (−/−) null mice have a defect in the beta-oxidation of fatty acids.

Accordingly, the level of ADMA and BHB are useful as diagnostic tools to assess patient outcome, both being elevated in the Cav-1 (−/−) null mice. According to the present invention, ADMA and BHB levels can be measured in patient serum/plasma, or directly determined from homogenates of fresh tumor tissue. Based upon the findings in Cav-1 (−/−) mice, high ADMA and BHB levels in breast cancer patient serum or human tumor samples is believed to correlate with poor clinical outcome. Prognostic tests may therefore be performed rapidly and quantitatively, allowing the identification and monitoring of high-risk cancer patients, both at diagnosis and during therapy. Determination of ADMA and BHB levels may also be used for treatment stratification.

We have also found that only a select number of miRs were transcriptionally upregulated in Cav-1 (−/−) stromal cells, miR-31 being the most significant (increased 4.2-fold). Based upon this findings in Cav-1 (−/−) mice, miR-31 is also a marker for poor patient outcome, allowing the identification and monitoring of high-risk cancer patients at diagnosis and during therapy. Determination of miR-31 level may also be used for treatment stratification.

For determining the level of ADMA, BHB or miR-31 in a sample, the sample may be, without limitation, a biological tissue or a biological fluid. The sample can be selected, without limitations, from excised breast tumor tissue, peripheral whole blood, and components thereof such as blood serum (“serum”) and blood plasma (“plasma”). In preferred embodiments, the sample is plasma. The sample is obtained from the subject using conventional methods in the art. For instance, one skilled in the art knows how to draw blood and how to process it in order to obtain serum and/or plasma for use in practicing the described methods.

While any elevated level of ADMA, BHB or miR-31 may be indicative of disease, according to some embodiments, the level is at least 1.5-fold, 2-fold, 2.5-fold, 3-fold, 3.5-fold or 4-fold greater than the level in a control.

The control sample in one embodiment is a biological material representative of healthy, cancer-free subjects, and/or cells or tissues. The level of ADMA, BHB or miR-31 in a control sample is desirably typical of the general population of normal, cancer-free subjects or of a particular individual at a particular time (e.g. before, during or after a treatment regimen), or in a particular tissue. The control sample can be removed from subject expressly for use in the methods described in this invention, or can be any biological material representative of normal, cancer-free subjects, including cancer-free biological material taken from the same subject outside the suspected cancerous lesion. The control sample can also refer to an established level of of ADMA, BHB or miR-31 representative of the cancer-free population, that has been previously established based on measurements from normal, cancer-free subjects.

Methods for quantitatively measuring the level of a biomarker such as ADMA, BHB or miR-31 in a tissue or a biological fluid are well known in the art. In some embodiments, assessing the level of ADMA, BHB or miR-31 involves the use of a detector molecule for the biomarker. Detector molecules can be obtained from commercial vendors or can be prepared using conventional methods in the art. Exemplary detector molecules include, but are not limited to, an antibody, aptamer or small molecule that binds specifically to ADMA, BHB or miR-31. Small molecules that bind specifically to any one of the biomarkers can be identified using conventional methods in the art, for instance, screening of compounds using combinatorial library methods known in the art, including biological libraries, spatially-addressable parallel solid phase or solution phase libraries, synthetic library methods requiring deconvolution, the “one-bead one-compound” library method, and synthetic library methods using affinity chromatography selection. Methods for preparing aptamers are also well-known in the art.

In some embodiments, the level of ADMA, BHB or miR-31 is assessed using an antibody. Thus, exemplary methods for assessing the level of ADMA, BHB or miR-31 in a biological fluid sample include various immunoassays, for example, immunohistochemistry assays, immunocytochemistry assays, ELISA, capture ELISA, sandwich assays, enzyme immunoassay, radioimmunoassay, fluorescence immunoassay, and the like, all of which are known to those of skill in the art. See e.g. Harlow et al., 1988, Antibodies: A Laboratory Manual, Cold Spring Harbor, N.Y.; Harlow et al., 1999, Using Antibodies: A Laboratory Manual, Cold Spring Harbor Laboratory Press, NY. Solid phase immunoassays can be particularly useful. Where two or more of the markers are assessed simultaneously, a panel of antibodies in an array format can be utilized. Custom antibody microarrays or chips can be obtained commercially. The generation of polyclonal antibodies is accomplished by inoculating the desired animal with an antigen and isolating antibodies which specifically bind the antigen therefrom.

Monoclonal antibodies directed against one biomarker identified herein may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115).

The level of ADMA, BHB or miR-31 may be determined by aptamer-based assays, which are very similar to antibody-based assays, but with the use of an aptamer instead of an antibody. An aptamer can be any polynucleotide, generally a RNA or a DNA, which has a useful biological activity in terms of biochemical activity or molecular recognition attributes. Usually, an aptamer has a molecular activity such as having an enzymatic activity or binding to a polypeptide at a specific region (i.e., similar to an epitope for an antibody) of the polypeptide.

Other methods for assessing the level of ADMA, BHB or miR-31 include chromatography (e.g., HPLC, gas chromatography, liquid chromatography), capillary electrophoresis and mass spectrometry. For instance, a chromatography medium comprising a cognate receptor for the biomarker, an aptamer that binds specifically to the biomarker, or a small molecule that binds specifically to the biomarker can be used to substantially isolate the biomarker from the sample of biological fluid.

ADMA may be quantified by high-performance liquid chromatography (HPLC). According to one such widely applied method, HPLC analysis is performed by extraction of samples with cation-exchange columns followed by o-phthalaldehyde derivatization and reversed-phase HPLC with fluorescence detection (Bode-Boger et al., Biochem Biophys Res Commun 1996; 219:598-603.). This basic method has been modified by several groups with respect to the extraction procedure (Chen et al., J Chromatogr B Biomed Sci Appl 1997; 692:467-71), the derivatization reagent (Marra et al., Anal Biochem 2003; 318:13-7), or the HPLC column used (Pettersson et al., J Chromatogr B Biomed Sci Appl 1997; 692:257-62). Other analytic procedures for ADMA that may be utilized include capillary electrophoresis (Causse et al., J Chromatogr B Biomed Sci Appl 2000; 741:777-83), liquid chromatography-mass spectrometry (Vishwanathan et al., J Chromatogr B Biomed Sci Appl 2000; 748:157-66; Martens-Lobenhoffer J et al., J Chromatogr B Anal Technol Biomed Life Sci 2003; 798:231-9), and gas chromatography-mass spectrometry (Tsikas et al., J Chromatogr B Anal Technol Biomed Life Sci 2003; 798:87-99; Albsmeier et al., J Chromatogr B Anal Technol Biomed Life Sci 2004; 809:59-65).

In another embodiment, ADMA is quantified by ELISA. One such ELISA technique for ADMA is reported by Schulze et al., Clin Chem Lab Med 2004; 42(12):1377-1383.

BHB may be quantified, for example, by enzymatic spectrophotometric assays. See e.g., Brashear et al., Analytical Biochemistry 1983; 131 (2):478-482. The most widely-used colorimetric test for BHB has been the reduction of the colorless dye 2-(4-indophenyl)-3-(4-nitrophenyl)-5-phenyltetrazolium chloride hydrate to a colored formazan compound. Another assay for BHB involves its oxidization to acetoacetic acid by beta.-hydroxybutyric acid dehydrogenase in the presence of nicotinamide adenine dinucleotide (NAD). This reaction produces reduced NAD (NAD-H), that in turn interacts with a tetrazolium dye to produce a colored formazan compound. The degree and intensity of the color transition then are correlated to the amount of DHBA in the test sample.

In another embodiment, the BHB assay method of U.S. Pat. No. 5,326,697 may be utilized. The method utilizes a reductive pathway based upon lipoamide dehydrogenase (LADH) and a thiol-sensitive indicator dye, such as Ellman's reagent, a derivative of Ellman's reagent or, preferably, a substituted isobenzothiazolone. After the DHBA has reacted with DHBA dehydrogenase and NAD to form NAD-H, LADH then interacts with the NAD-H and D,L-lipoamide to form a thiol compound, 6,8-dimercaptooctamide. The 6,8-dimercaptooctamide then interacts with a thiol-responsive indicator dye, such as Ellman's reagent, a derivative of Ellman's reagent or a suitable isobenzothiazolone. Upon interaction with the thiol compound, the thiol-responsive indicator dye undergoes a detectable or measurable color transition that can be correlated to the amount of BHB in the test sample.

The level of BHB may also be determined radioisotopically. A radioisotopic procedure for the assay of 3-hydroxybutyrate in the picomolar range is based on the measurement of NADH, generated in the dehydrogenase reaction, through the conversion of 2-[U-¹⁴C]ketoglutarate to ¹⁴C-labeled L-glutamate in the presence of beef liver glutamate dehydrogenase (Ramirez et al., Biochemical Medicine and Metabolic Biology 1991; (2):227-234.

In another embodiment, levels of ADMA and BHB in patient samples may be determined by sample solvent extraction, chromatographic separation of sample components, and mass spectrometry. The sample signature is compared with the signature of ADMA and BHB and quantitative values are obtained.

The level of miR-31 in a tissue sample may advantageously be determined by conventional quantitative polynucleotide assay methods. The nucleotide sequence of miR-31, as provided by GenBank (ACCESSION NR_{—}029505; VERSION NR_{—}029505.1; GI:262205714), is as follows:

(SEQ ID NO: 1)
GGAGAGGAGG CAAGATGCTG GCATAGCTGT TGAACTGGGA
ACCTGCTATG CCAACATATT GCCATCTTTC C.

Such quantitative polynucleotide assay methods include, for example, polymerase chain reaction analyses, Northern analyses, and probe arrays. Any RNA isolation technique that does not select against the isolation of miR can be utilized for the purification of RNA from samples In some embodiments, the RNA sample may be depleted of one or more RNAs, for example, an RNA sample depleted of rRNA. General methods for total RNA extraction are well known in the art and are disclosed in standard textbooks on molecular biology. The practice of the invention is not limited to any one method of mRNA detection or quantification recited herein, but rather encompasses all presently known or heretofore unknown methods.

In another embodiment niR-31 is quantified using an amplification (e.g., PCR) assay. The target transcript nucleotide sequence act as a template in an amplification reaction. In a quantitative amplification, the amount of amplification product will be proportional to the amount of template in the original sample. Comparison to appropriate controls provides a measure of the transcript level.

### Kits

The invention also relates to kits for prognosis and disease progression monitoring, as described above. A kit comprises a set of reagents that specifically detects the level of ADMA, BHB or miR-31, and instructions for using the kit for prognosis of breast cancer, or monitoring the progress of disease.

In some embodiments, the set of reagents comprises antibodies or aptamers that specifically bind to the ADMA, BHB or miR-31. For example, the kit can comprise an antibody, an antibody derivative, or an antibody fragment that binds specifically with ADMA, BHB or miR-31. Such kits may also comprise a plurality of antibodies, antibody derivatives, or antibody fragments wherein the plurality of such antibody agents binds specifically with a marker protein, or a fragment of the protein.

For antibody-based kits, the kit can comprise, for example: (1) a first antibody (e.g., attached to a solid support) that binds to a marker protein; and, optionally, (2) a second, different antibody that binds to either the protein or the first antibody and is conjugated to a detectable label.

The kit can further comprise components necessary for detecting the detectable label (e.g., an enzyme or a substrate).

Kits for practice of the invention may also comprise, e.g., buffering agents, preservatives, or protein stabilizing agents. The kit can also contain a control sample or a series of control samples which can be assayed and compared to the test sample. Each component of the kit can be enclosed within an individual container and all of the various containers can be within a single package, along with instructions for interpreting the results of the assays performed using the kit.

### Disease Progression Monitoring

In another embodiment, the invention provides a method of monitoring the progression of breast cancer in a subject. Samples are obtained from the subject at different time points and analyzed as described above. An elevated level of ADMA, BHB or miR-31 in a later sample relative to an earlier sample is an indication that the breast cancer has progressed in the patient.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Treatment

In one embodiment, breast cancer is treated by administering an effective amount of an inhibitor of ADMA or BHB. In another embodiment, breast cancer is treated by administering and effective amount of an inhibitor of ADMA production, or of ketone production or re-utilization to halt tumor growth, leading to tumor regression. As such, the enzymes associated with (1) ADMA production (all PRMT family members), (2) ketone production (ACLY, HMGCS1/2, HMGCL, HMGCLL1 and BDH1/2) and (3) ketone re-utilization (ACAT1/2 and OXCT1/2) are targets for inhibition for cancer therapy.

### EXAMPLES

### Material and Methods

### Animals

All animals were housed and maintained in a pathogen-free environment/barrier facility according to the guidelines of the National Institutes of Health. Mice were kept on a 12-hour light/dark cycle with ad libitum access to chow and water. Cav-1 (−/−) deficient mice were generated, as we previously described (Razani et al., J Biol Chem 2001; 276:38121-38). All mice used for metabolomics analysis were in the FVB/N genetic background.

### Metabolomics Analysis and Metabolite Identification

Metabolomic profiling analysis was achieved with the services of Metabolon, Inc., using the following protocol, as described in Lawton et al., Pharmacogenomics 2008; 9:383-97 and Ryals et al., Pharmacogenomics 2007; 8:863-6. Unbiased metabolic profiling technology based on sample extraction and mass spectrometry was applied to the (1) mammary fat pads and (2) lung tissue samples, from virgin female 5-month-old WT and Cav-1 (−/−) null mice (n=6 tissues samples, for each organ type and for each genotype, for a total of 24 samples). To efficiently recover various metabolites, the sample preparation consisted of sequential organic and aqueous extractions. Four solvent extraction steps were used: 400 μl tridecanoic acid (2.5 mg/mL) in ethyl acetate:ethyl alcohol (1:1), 200 μl methanol, 200 μl methanol:H2O (3:1) and 200 μl dichloromethane:methanol (1:1). The resulting pooled extract was equally divided into a liquid chromatography (LC) fraction and a gas chromatography (GC) fraction. After chromatographic separation, a full-scan mass spectra was carried out to record all detectable ions present in the samples. In both the GC and LC methods, we used internal standards to calibrate retention times of metabolites across all of the samples in the study and for quality control on each instrument run. Known chemical structure metabolites were identified by matching the ions' chromatographic retention index and mass spectra fragmentation signatures, with Metabolon, Inc.'s reference library entries created using Metabolon's software. Each entry identified by this software is visually inspected with VPhil Software to confirm the acceptance of that metabolite in the study. Peptides were identified using standard tandem MS sequencing techniques (Kinter et al., Eds. Protein Sequencing and Identification Using Tandem Mass Spectrometry. New York: John Wiley & Sons Inc. 2000. Missing values for a specific metabolite were assumed to have fallen below the limits of detection and were imputed with the observed minimum. Quantitative values were derived from integrated raw detector counts of the mass spectrometers. For the convenience of data visualization, the raw area counts for each biochemical were resealed by dividing each sample's value by the median value for the specific biochemical. Statistical analysis of the data was performed using JMP (SAS, www<<dot>>jmp<<dot>>com), commercial software package and “R” (cran<<dot>>r-project<<dot>>org), which is a freely available, open-source software package. A log transform was applied to the observed relative concentrations for each biochemical because, in general, the variance increased as a function of a biochemical's average response. To account for multiple hypothesis testing, we estimated the false discovery rate (Benjamini et al., J R Soc Series B 1995; 57:289-300) with the q-value method (Storey et al., Proc Natl Acad Sci USA 2003; 100:9440-5). Data analyses were carried out by JMP (SAS Institute, Inc., Cary, N.C.) and R (R Foundation for Statistical Computing, Vienna, Austria). Significance was defined at p≦0.05 and q≦0.1.

Micro RNA (miR) Gene Chip Profiling.

The FlashTag™ Biotin RNA Labeling Kit (Genisphere, LLC, Hatfield, Pa.) was used, according to the manufacturer's protocol, to generate labeled miRNA molecules from 500 ng of total RNA by poly (A) tailing followed by ligation of the biotinylated signal molecule to the target RNA sample. Biotin labeled sample was hybridized to Affymetrix® GeneChip® miRNA Arrays (Affymetrix, Santa Clara, Calif.), Sanger miRBase V11. Hybridized chips were washed and stained using GeneChip® Fluidic Station 450 and scanned on an Affymetrix GeneChip® Scanner 3000, using Command Console Software 3.1. Affymetrix miRNA QCtool (V1.0.33.0) was used for data summarization and quality control. Background correction and normalization were done using Robust Multichip Average (RMA), baseline to median of all samples, analysis were performed with GeneSpring GX V 10.0 software (Agilent, Palo Alto, Calif.).

### Example 1

### Metabolomic Analysis of Cav-1 (−/−) Null Tissues from Mammary Fat Pads

Mammary fat pads were harvested from age-matched female WT and Cav-1 (−/−) null mice (n=6 for each genotype) and subjected to an unbiased metabolomic analysis. Over 200 known compounds were identified by mass spectrometry analysis and their levels were quantitated. Interestingly, a large number of compounds were significantly changed in Cav-i (−/−) mammary fat pads (n=103; 92 UP; 11 DOWN), consistent with a severe metabolic phenotype. See Table 1.

In Table 1, fold-changes (knock out/wild type) are shown in parentheses after each metabolite that showed a significant change (p≦0.05). In one case, an asterisk (*) indicates p≦0.1. Metabolites showing an increase of 2.5 or greater are underlined. All other p values were p≦0.05.

Table 1. Metabolomic Analysis of Mammary Fat Pads from Cav-1 (−/−) Deficient Mice

### (A) Amino Acids:

(1) Alanine and aspartate metabolism: alanine (1.7); asparagine (2.3); aspartate (2.7)

(2) Cysteine, methionine, SAM, taurine metabolism: cysteine (1.6); hypotaurine (1.8); methionine (2.2); N-acetylmethionine (2.7); S-adenosylhomocysteine (SAH) (0.7)

(3) Glutamate metabolism: glutamate (1.9); glutamine (1.6); N-acetyl-aspartyl-glutamate (NAAG) (1.6)

(4) Glutathione metabolism: 5-oxoproline (1.5); cysteine-glutathione disulfide (1.4); glutathione, oxidized (GSSG) (1.5); glutathione, reduced (GSH) (1.7)

(5) Glycine, serine and threonine metabolism: glycine (2.3); serine (2.5); threonine (2.0)

(6) Histidine metabolism: histamine (2.5); histidine (2.4); urocanate (3.0)

(7) Lysine metabolism: lysine (1.7); pipecolate (1.9)

(8) Phenylalanine & tyrosine metabolism: phenylalanine (2.3); tyrosine (2.5)

(9) Tryptophan metabolism: 5-hydroxyindoleacetate (2.7); C-glycosyl-tryptophan (1.7); tryptophan (2.3).

(10) Urea cycle; arginine-, proline-, metabolism: arginine (1.9); assymetric dimethylarginine (ADMA) (3.3); proline (2.3); trans-4-hydroxyproline (2.0); urea (2.5)

(11) Valine, leucine and isoleucine metabolism: isoleucine (2.5); leucine (2.3); valine (2.0)

### (B) Peptides:

(7) Dipeptide: glycylproline (2.8); proline-hydroxy-proline (1.8)

(8) Gamma-glutamyl: gamma-glutamylglutamate (1.7); gamma-glutamylisoleucine (2.0); gamma-glutamylleucine (1.9); gamma-glutamylphenylalanine (1.8); gamma-glutamyltryptophan (2.1); gamma-glutamyltyrosine (1.9); gamma-glutamylvaline (1.7)

### (C) Carbohydrates:

(9) Aminosugars metabolism: N-acetylglucosamine 6-phosphate (1.6)

(10) Fructose, mannose, galactose, starch and sucrose metabolism: erythrose (2.0); fructose (2.0); mannose-6-phosphate (1.8)

(11) Glycolysis, gluconeogenesis, pyruvate metabolism: fructose-6-phosphate (1.9); glucose (1.5); glucose-6-phosphate (G6P) (1.9); pyruvate (1.4)*

(12) Nucleotide sugars, pentose metabolism: ribose (1.9); sedoheptulose-7-phosphate (1.7)

(13) Sugar alcohol: myo-inositol (1.4)

### (D) Cofactors and Vitamins:

(14) Ascorbate and aldarate metabolism: ascorbate (Vitamin C) (11.2); threonate (1.4)

(15) Nicotinate and nicotinamide metabolism: nicotinamide (1.3)

(16) Pentothenate and CoA metabolism: pantothenate (1.4)

(17) Riboflavin metabolism: riboflavin (Vitamin B2) (2.6)

(18) Tocopherol metabolism: alpha-tocopherol (2.7)

(19) Vitamin B6 metabolism: pyridoxate (1.8)

### (E) Energy:

(20) Krebs cycle: fumarate (1.6); malate (1.4)

(21) Oxidative phosphorylation: methylphosphate (1.6); phosphate (1.3) (F) Lipids:

(22) Carnitine metabolism: 3-dehydrocarnitine (1.3); carnitine (0.9); malonylcarnitine (0.6)

(23) Essential fatty acid: dihomo-linolenate (20:3n3 or n6) (1.5)

(24) Fatty acid, ester: n-butyl oleate (1.5)

(25) Glycerolipid metabolism: choline (1.3); ethanolamine (2.1) glycerol (1.2); phosphoethanolamine (2.4)

(26) Inositol metabolism: inositol 1-phosphate (I1P) (2.4)

(27) Ketone bodies: 3-hydroxybutyrate (BHBA) (4.3)

(28) Long chain fatty acids: arachidonate (20:4n6) (1.5); margarate (17:0) (1.4); myristoleate (14:1n5) (1.8)

(29) Monoacylglycerol: 1-stearoylglycerol (1-monostearin) (1.4)

(30) Sterol/Steroid: cholesterol (1.6)

### (G) Nucleotides:

(31) Purine metabolism, (hypo)xanthine/inosine containing: hypoxanthine (1.6); inosine (0.7); xanthine (1.7); xanthosine (1.3)

(32) Purine metabolism, adenine containing: adenosine 2′-monophosphate (2′-AMP) (2.4); N1-methyladenosine (2.0)

(33) Purine metabolism, guanine containing: guanosine (0.4)

(34) Purine metabolism, urate metabolism: urate (0.7)

(35) Pyrimidine metabolism, cytidine containing: 2′-deoxycytidine (2.2); cytidine (2.5); cytidine 5′-monophosphate (5′-CMP) (0.8); cytidine-3′-monophosphate (3′-CMP) (1.9)

(36) Pyrimidine metabolism, uracil containing: pseudouridine (1.7); uracil (3.8)

### (H) Xenobiotics:

(37) Benzoate metabolism: 4-ethylphenylsulfate (2.1); catechol sulfate (2.5); hippurate (1.8).

### Example 2

### Metabolomic Analysis of Cav-1 (−/−) Null Tissues from Lung Tissue

We also independently compared our results from the mammary fat pad with lung tissue, as adipose tissue and lung tissue express the highest levels of Cav-1. Only concordant changes were selected and are shown in Table 2. ADMA, pyruvate and BHB were significantly elevated in lung tissue, consistent with the idea that Cav-1 (−/−) null tissues are undergoing (1) oxidative stress and (2) mitochondrial dysfunction. Box plots for ADMA and BHB are shown in FIG. 1.

In Table 2, only shown are metabolites and fold-changes of knock out to wild-type (KO/WT) showing concordant changes in both the mammary fat pad and lung tissue. An asterisk (*) indicates p≦0.1. All other p values were p≦0.05.

TABLE 2
Metabolomic analysis of mammary fat pads and lug
tissue from Cav-1 (−/−) deficient mice

| | Mammary | Lung |
|---|---|---|
| Metabolites | (KO/WT) | (KO/WT) |
| pipecolate | 1.9 | 1.3 |
| assymetric dimethylarginine (ADMA) | 3.3 | 1.4 |
| glycylproline | 2.8 | 1.4 |
| pyruvate | 1.4* | 1.9 |
| carnitine | 0.9 | 0.9 |
| dihomo-linolenate (20:3n3 or n6) | 1.5 | 1.3 |
| 3-hydroxybutyrate (BHBA) | 4.3 | 3.5 |
| inosine | 0.7 | 0.7 |
| cytidine-3′-monophosphate (3′-CMP) | 1.9 | 1.3 |
| 4-ethylphenylsulfate | 2.1 | 2.2 |

### Example 3

### Micro-RNA (miR) Profiling of Cav-1 (−/−) Stromal Cells

Cav-1 (−/−) stromal cells were subjected to miR-profiling as described in Materials and Methods. The results, shown in Table 3. demonstrate that only a select number of miRs were transcriptionally upregulated in Cav-1 (−/−) stromal cells. For this analysis, we chose a cut-off of 1.5-fold increased (KO/WT). P-values are as shown. In Table 3, “ns” means not significant. miR-31 and miR-34c showed the most significant p-values. Notably, miR-31 and miR-34c were increased 4.2-fold and nearly three-fold, respectively.

TABLE 3
Upregulation of miR's in Cav-1 (−/−) null stromal cells

| Symbol | Fold change (KO/WT) | p-value |
|---|---|---|
| miR-31 | 4.24 | 0.002 |
| miR-34c | 2.95 | 0.01 |
| miR-423-3p | 2.18 | 0.02 |
| miR-193b | 2.08 | 0.09/ns |
| miR-423-5p | 1.99 | 0.03 |
| miR-342-5p | 1.96 | 0.05 |
| miR-210 | 1.74 | 0.07/ns |
| miR-574-3p | 1.72 | 0.02 |
| miR-182 | 1.71 | 0.04 |
| miR-298 | 1.71 | 0.04 |
| miR-28 | 1.70 | 0.1/ns |
| miR-744 | 1.68 | 0.1/ns |
| miR-20b | 1.62 | 0.1/ns |
| miR-467h | 1.59 | 0.07/ns |
| miR-185 | 1.58 | 0.04 |
| miR-222 | 1.55 | 0.04 |
| miR-125a-5p | 1.53 | 0.06/ns |

### Example 4

### ADMA and Ketone Metabolism in Cav-1 (−/−) Stromal Cells and Human Tumor Stroma

To further validate the observations that ADMA and BHB are the two major metabolites that increased in mammary fat pads and are reflective of oxidative stress and mitochondrial dysfunction in Cav-1 (−/−) stroma, we analyzed transcriptional profiling data for the expression of the relevant enzymes that are involved in ADMA and ketone metabolism. Both transcriptional profiles from Cav-1 (−/−) null stromal cells and human breast cancer tumor stroma were analyzed in parallel and are presented in Table 4. We analyzed the mRNA expression of the genes involved in ADMA production (PRMT gene family members) and degradation (DDAH1/2), as well as nitric oxide (NO) related genes, as ADMA drives NOS uncoupling and the production of superoxide, instead of NO. The results show that the genes involved in both ADMA production (PRMT2/7/8) and degradation (DDAH1/2), as well as nitric oxide production (NOS1/2/3 or NOS trafficking), are all transcriptionally overexpressed, both in human tumor stroma and in Cav-1 (−/−) stromal cells.

In Table 4, for ADMA production, only PRMT genes that were upregulated in the tumor stromal gene sets or Cav-1 KO MSCs are listed. (MSCs=mesenchymal stem cells.)

TABLE 4
Transcriptional profiling of human breast cancer
tumor stroma: ADMA and BHB metabolism

| | | | | Cav-1 KO MSCs | |
|---|---|---|---|---|---|
| | Tumor | Recurrence | Metastasis | Fold | |
| Gene symbol | stroma | stroma | stroma | Change | p-value |

ADMA Production

| | | | | | |
|---|---|---|---|---|---|
| PRMT2 | | 1.35SE−02 | | 1.68 | 0.1 |
| PRMT7 | | | 1.76E−02 | | |
| PRMT8 | 2.41E−18 | | 3.04E−02 | 1.56 | 0.02 |

ADMA Degradation

| | | | |
|---|---|---|---|
| DDAH1 | 2.36E−10 | 1.90 | 0.006 |
| DDAH2 | 7.25E−16 | 1.46 | 0.005 |

Ketone Production

| | | | | | |
|---|---|---|---|---|---|
| ACLY | 1.55E−02 | 2.50E−02 | | 2.19 | 0.02 |
| HMGCS1 | | | | 1.34 | 0.04 |
| HMGCS2 | 3.91E−193 | 30E−02 | | 1.52 | 0.04 |
| HMGCL | | | | 2.24 | 0.03 |
| HMGCLL1 | 3.19E−13 | 5.95E−04 | | | |
| BDH1 | 1.04E−06 | 6.46E−03 | | 1.38 | 0.04 |
| BDH2 | | | 3.54E−02 | | |

Ketone Re-Utilization

| | | |
|---|---|---|
| ACAT1 | | |
| ACAT2 | 1.57 | 0.1 |
| OXCT1 | 1.25 | 0.04 |
| OXCT2 | 1.38 | 0.05 |

Nitric Oxide Production

| | | | | | |
|---|---|---|---|---|---|
| NOS1 | 2.02E−13 | 1.82E−04 | | | |
| NOS2 | | | | 1.36 | 0.05 |
| NOS3 | 1.36E−06 | 1.21E−02 | | 1.65 | 0.05 |
| NOSIP | | | 3.04E−02 | 1.59 | 0.03 |
| NOSTRIN | 2.09E−04 | | | 1.21 | 0.02 |

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

We claim:

1. A prognostic method for breast cancer in a subject comprising:
(a) determining the level of at least one of the following in a test sample from a subject: (i) asymmetric dimethyl arginine; (ii) beta-hydroxybutyrate; and (iii) miR-31; and
b) comparing the level of at least one of (i) asymmetric dimethyl arginine, (ii) beta-hydroxybutyrate, and (iii) miR-31 in said test sample to the level of in a control sample, wherein an elevated level of at least one of (i) asymmetric dimethyl arginine, (ii) beta-hydroxybutyrate, and (iii) miR-31 in said test sample relative to the level in said control sample is a prognostic indicator of the course of breast cancer disease in said subject.

2. A method of monitoring the progression of breast cancer in a subject, said method comprising:
(a) obtaining a first sample from a subject at a first time point and a second sample from said subject at a second time point;
(b) determining the level of at least one of the following in said first and second samples: (i) asymmetric dimethyl arginine; (ii) beta-hydroxybutyrate; and (iii) miR-31; and
(c) comparing the level of said at least one of (i) asymmetric dimethyl arginine, (ii) beta-hydroxybutyrate, and (iii) miR-31 in said first sample to the level in said second sample, wherein an elevated level of at least one of (i) asymmetric dimethyl arginine, (ii) beta-hydroxybutyrate, and (iii) miR-31 in said second sample relative to the level in said first sample is an indication that the cancer has progressed in said subject.

3. The method of claim 1 or 2 wherein the level of asymmetric dimethyl arginine is determined.

4. The method of claim 1 or 2 wherein the level of beta-hydroxybutyrate is determined.

5. The method of claim 1 or 2 wherein the level of miR-31 is determined.

6. The method of claim 1 or 2 wherein the sample comprises serum or plasma.

7. The method of claim 1 or 2 wherein the sample comprises excised tumor tissue.

8. A method for treating breast cancer comprising administering to a breast cancer patient in need of such treatment an effective amount of an inhibitor of asymmetric dimethyl arginine or an inhibitor of beta-hydroxybutyrate.

9. The method according to claim 8 comprising administering to the patient an effective amount of an inhibitor of asymmetric dimethyl arginine.

10. The method according to claim 8 comprising administering to the patient an effective amount of an inhibitor of beta-hydroxybutyrate.


SEQUENCE LISTING (1 sequence)

SEQ ID NO: 1 - DNA, 71 bp, Homo sapiens
ggagaggaggcaagatgctggcatagctgttgaactgggaacctgctatgccaacatattgccatctttcc